(19) United States
(12) Patent Application Publication
(10) Pub. No.: US 2008/0070885 A1
(43) Pub. Date: Mar. 20, 2008

(54) MACROCYCLIC COMPOUNDS HAVING ASPARTIC PROTEASE INHIBITING ACTIVITY AND PHARMACEUTICAL USES THEREOF

(76) Inventors: Claudia Betschart, Basel (CH); Marina Tintelnot-Blomley, Maulburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/939,936

(22) Filed: Nov. 14, 2007

Related U.S. Application Data
(63) Continuation of application No. 10/561,949, filed on Dec. 22, 2005, now abandoned.

(30) Foreign Application Priority Data
Jul. 3, 2003 (GB) 0315654.4

(51) Int. Cl. A61K 31/395; A61P 25/00; A61P 9/00; C07D 245/04; C07D 281/00

(52) U.S. Cl. 514/183; 540/468; 540/472

(57) ABSTRACT

The present invention relates to macro-cyclic compounds of formula (I), wherein R₁, is (C₁₋₈)alkyl, (C₁₋₄)alkoxy(C₁₋₄)alkyl, hydroxy(C₁₋₆)alkyl, (C₁₋₄)alkylthio(C₁₋₄)alkyl, (C₁₋₆)alkenyl, (C₃₋₇)cycloalkyl, (C₃₋₇)cycloalkyl(C₁₋₄)alkyl, piperidinyl or pyrrolidinyl, R₂ and R₄, independently, are hydrogen or optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl or heteroaryl(C₁₋₄)alkyl, or R₂ and R₄, together with the nitrogen to which they are attached, form an optionally substituted piperidino, pyrrolidinyl, morpholino or piperazinyl group, R₃ is hydrogen or (C₁₋₄)alkyl, X₁ is CH₂, X₂ is CH₂, O, S, CO, COO, OCO, NHCO, CONH, or NR, R being hydrogen or (C₁₋₄)alkyl, Y is (C₁₋₈)alkylen or (C₁₋₈)alkylenoxy(C₁₋₆)alkylen, (C₁₋₈)alkenylen or (C₁₋₈)alkenylenoxy(C₁₋₆)alkylen, Ar is a phenyl ring optionally mono- di or trisubstituted by, independently, hydroxy or halogen, whereby X₁, and X₂ are in meta or para position to each other, and either Z is CO, AA is a natural or unnatural alpha-amino-acid, and n is 0 or 1, or Z is S0₂, AA is an optionally substituted ethylencarbonyl group (derived from a natural or unnatural alpha-amino acid by replacement of the nitrogen by a methylen group), and n is 1; processes for the preparation of these compounds; pharmaceutical compositions and combinations comprising the same; and their use in the treatment of neurological and vascular disorders related to beta-amyloid generation and/or aggregation.

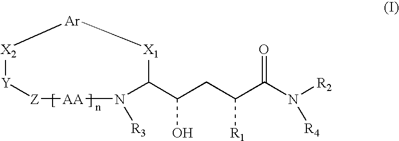

The present invention relates to novel macrocyclic compounds, their preparation, their use as pharmaceuticals and pharmaceutical compositions containing them.

More particularly the invention provides compounds of formula I 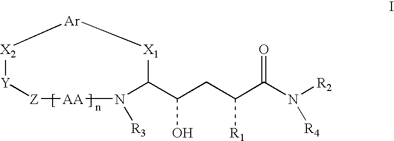
wherein
R₁ is (C₁₋₈)alkyl, (C₁₋₄)alkoxy(C₁₋₄)alkyl, hydroxy(C₁₋₆)alkyl, (C₁₋₄)alkylthio(C₁₋₄)alkyl, (C₁₋₆)alkenyl, (C₃₋₇)cycloalkyl, (C₃₋₇)cycloalkyl(C₁₋₄)alkyl, piperidinyl or pyrrolidinyl,
R₂ and R₄, independently, are hydrogen or optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl or heteroaryl(C₁₋₄)alkyl, or
R₂ and R₄, together with the nitrogen to which they are attached, form an optionally substituted piperidino, pyrrolidinyl, morpholino or piperazinyl group,
R₃ is hydrogen or (C₁₋₄)alkyl,
X₁ is CH₂,
X₂ is CH₂, O, S, CO, COO, OCO, NHCO, CONH, or NR, R being hydrogen or (C₁₋₄)alkyl,
Y is (C₁₋₈)alkylen or (C₁₋₈)alkylenoxy(C₁₋₆)alkylen, (C₁₋₈)alkenylen or (C₁₋₈)alkenylenoxy (C₁₋₆)alkylen,
Ar is a phenyl ring optionally mono- di- or trisubstituted by, independently, hydroxy or halogen, whereby X₁ and X₂ are in meta or para position to each other,
   and either
Z is CO,
AA is a natural or unnatural alpha-amino-acid, and
n is 0 or 1,
   or
Z is SO₂,
AA is an optionally substituted ethylencarbonyl group (derived from a natural or unnatural alpha-amino acid by replacement of the nitrogen by a methylen group), and
n is 1
   in free base or acid addition salt form.

Halogen denotes fluorine, bromine, chlorine or iodine.

When R₂ and/or R₄ is substituted alkyl or cycloalkyl, or together with the nitrogen to which they are attached, form a substituted piperidino, pyrrolidinyl, morpholino or piperazinyl group, substituents may be one to three groups selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonylamino, (C₁₋₄)alkylcarbonyl, (C₁₋₄)sulfonyl, cyano, oxo, hetero (C₃₋₇)cycloalkyl or heteroaryl.

When R₂ and/or R₄ is substituted aryl or heteroaryl, substituents may be one to three groups selected from halogen, hydroxy, cyano, trifluoromethyl, carboxy, (C₁₋₄)alkyloxycarbonyl, (C₁₋₄)alkylcarbamoyl, (C₁₋₄)alkylsulfonyl, (C₁₋₄)alkylcarbonyloxy, (C₁₋₄)alkylcarbonyl, (C₁₋₄)alkyl, (C₁₋₄)alkoxy or hydroxy(C₁₋₄)alkyl.

Aryl is an aromatic 6-membered ring optionally mono-, di- or tri-substituted by, independently, hydroxy, cyano, trifluoromethyl, carboxy, (C₁₋₄)alkyloxycarbonyl, (C₁₋₄)alkylcarbamoyl, (C₁₋₄)alkylsulfonyl, (C₁₋₄)alkylcarbonyloxy, (C₁₋₄)alkylcarbonylamino, (C₁₋₄)alkylcarbonyl, (C₁₋₄)alkyl, (C₁₋₄)alkoxy or hydroxy(C₁₋₄)alkyl. It can also be fused with an cycloalkyl or additional aromatic or heteroaromatic ring (e.g. to form a naphthyl, quinolinyl, indolyl group).

Heteroaryl is an aromatic 5- or 6-membered ring in which 1, 2 or 3 atoms are heteroatoms independently selected from O, N and S, optionally mono- di- or tri-substituted by, independently, hydroxy or halogen. Heteroaryl is for example 1-methyl-1H-pyrrol-2-yl or 1H-imidazol-2-yl. It can also be fused with an cycloalkyl or additional aromatic or heteroaromatic ring (e.g. to form a quinolinyl, indolyl group).

Any alkyl or alkoxy group is straight or branched and is preferably methyl or methoxy.

In formula I the following significances are preferred independently, collectively or in any combination or sub-combination:

R₁ is preferably (C₁₋₈)alkyl, more preferably (C₁₋₄)alkyl, in particular methyl, ethyl or propyl.

R₂ is preferably (C₁₋₈)alkyl, more preferably (C₁₋₆)alkyl, in particular methyl, ethyl or propyl, or butyl, such as n-butyl, sec-butyl or tert-butyl.

R₃ is preferably hydrogen.

R₄ is preferably hydrogen.

X₂ is preferably methylen or oxygen.

Y is preferably (C₁₋₈)alkylen, more preferably (C₃₋₆)alkylen.

Ar is preferably unsubstituted phenylen whereby X₁ and X₂ are in meta position to each other.

AA is preferably selected from —N(H)—CH(CH₃)—C(O)—, —CH₂—CH(CH₃)—C(O)— and —CH₂—CH₂—C(O)—.

A preferred embodiment of the present invention relates to compounds of formula I, wherein
R₁ is (C₁₋₈)alkyl, (C₁₋₄)alkoxy(C₁₋₄)alkyl, hydroxy(C₁₋₆)alkyl, (C₁₋₄)alkylthio(C₁₋₄)alkyl, (C₁₋₆)alkenyl, (C₃₋₇)cycloalkyl, (C₃₋₇)cycloalkyl(C₁₋₄)alkyl, piperidinyl or pyrrolidinyl,
R₂ and R₄, independently, are
   (a) hydrogen
   (b) (C₁₋₈)alkyl, (C₃₋₇)cycloalkyl or (C₃₋₇)cycloalkyl(C₁₋₄)alkyl, in each case optionally substituted by one to three groups selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonylamino, (C₁₋₄)alkylcarbonyl, (C₁₋₄)sulfonyl, cyano, oxo, hetero(C₃₋₇)cycloalkyl or heteroaryl, or
   (c) aryl, aryl(C₁₋₄)alkyl, heteroaryl or heteroaryl(C₁₋₄)alkyl, wherein in the latter two radicals heteroaryl denotes an aromatic 5- or 6-membered ring in which 1, 2 or 3 atoms are heteroatoms independently selected from O, N and S, wherein all radicals are optionally substituted by one to three groups selected from halogen, hydroxy, cyano, trifluoromethyl, carboxy, (C₁₋₄)alkyloxycarbonyl, (C₁₋₄)alkylcarbamoyl, (C₁₋₄)alkylsulfonyl, (C₁₋₄)alkylcarbonyloxy, (C₁₋₄)alkylcarbonyl, (C₁₋₄)alkyl, (C₁₋₄)alkoxy or hydroxy(C₁₋₄)alkyl, or
R₂ and R₄, together with the nitrogen to which they are attached, form an piperidino, pyrrolidinyl, morpholino or piperazinyl group, each of which is optionally substituted by one to three groups selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonylamino, (C₁₋₄)alkylcarbonyl, (C₁₋₄)sulfonyl, cyano, oxo, hetero (C₃₋₇)cycloalkyl or heteroaryl,
R₃ is hydrogen or (C₁₋₄)alkyl,
X₁ is CH₂,
X₂ is CH₂, O, S, CO, COO, OCO, NHCO, CONH, or NR, R being hydrogen or (C₁₋₄)alkyl,
Y is (C₁₋₈)alkylen or (C₁₋₈)alkylenoxy(C₁₋₆)alkylen, (C₁₋₈)alkenylen or (C₁₋₈)alkenylenoxy(C₁₋₈)alkylen,
Ar is a phenyl ring optionally mono- di- or trisubstituted by, independently, hydroxy or halogen, whereby X₁ and X₂ are in meta or para position to each other,
   and either
Z is CO,
AA is a natural or unnatural alpha-amino-acid, and
n is 0 or 1,
   or
Z is SO₂,
AA is an optionally substituted ethylencarbonyl group (derived from a natural or unnatural alpha-amino acid by replacement of the nitrogen by a methylen group), and
n is 1.

Especially preferred are compounds of formula I, wherein
R₁ is (C₁₋₄)alkyl,
R₂ is (C₁₋₆)alkyl,
R₃ is hydrogen or (C₁₋₄)alkyl,
R₄ is hydrogen,
X₁ is CH₂,
X₂ is CH₂ or O,
Y is (C₁₋₈)alkylen,
Ar is unsubstituted phenylen, whereby X₁ and X₂ are in meta position to each other,
   and either
Z is CO,
AA is a natural or unnatural alpha-amino-acid, and
n is 0 or 1,
   or
Z is SO₂,
AA is an optionally substituted ethylencarbonyl group (derived from a natural or unnatural alpha-amino acid by replacement of the nitrogen by a methylen group), and
n is 1.

More preferred are compounds of formula I, wherein
R₁ is (C₁₋₄)alkyl,
R₂ is (C₁₋₆)alkyl,
R₃ is hydrogen or (C₁₋₄)alkyl,
R₄ is hydrogen,
X₁ is CH₂,
X₂ is CH₂ or O,
Y is (C₃₋₆)alkylen,
Ar is unsubstituted phenylen whereby X₁ and X₂ are in meta position to each other,
   and either
Z is CO,
AA is —N(H)—CH(CH₃)ₘ—C(O)—, wherein m is 0 or 1 and
n is 0 or 1,
   or
Z is SO₂,
AA is —CH₂—CH(CH₃)—C(O)— or —CH₂—CH₂—C(O)— and
n is 1.

Most preferred are those compounds as described in the Examples.

In a further aspect, the invention provides a process for the production of the compounds of formula I and their salts, comprising the steps of

a) for the production of a compound of formula I wherein Z is CO, cyclisation by amide formation of a compound of formula II 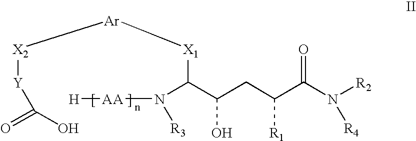
wherein R₁, R₂, R₃, R₄, X₁, X₂, Y, Ar, AA and n are as defined above,

b) for the production of a compound of formula I wherein Z is SO₂ and Y is (C₁₋₈)alkenylen or (C₁₋₈)alkenylenoxy(C₁₋₆)alkylen, cyclisation by metathesis of a compound of formula III 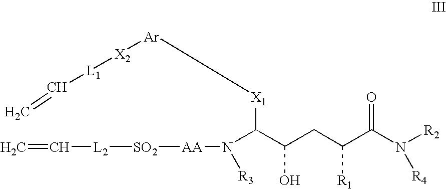
wherein R₁, R₂, R₃, R₄, X₁, X₂, Ar and AA are as defined above and L₁ and L₂, independently are alkylen or alkylenoxyalkylen groups, or

c) for the production of a compound of formula I wherein Z is SO₂ and Y is (C₁₋₈)alkylen or

(C₁₋₈)alkylenoxy(C₁₋₆)alkylen, hydrogenation of a compound of formula I wherein Z is SO₂ and Y is

(C₁₋₈)alkenylen or (C₁₋₈)alkenylenoxy(C₁₋₆)alkylen,

and recovering the so obtained compound of formula I in free base or acid addition salt form.

The reactions can be effected according to known methods, for example the cyclisation of process A can be effected as described in Example 1.

Working-up the reaction mixtures and purification of the compounds thus obtained may be carried out in accordance to known procedures.

Acid addition salts may be produced from the free bases in known manner, and vice-versa.

The starting material of formula II may be produced for example as described in Example 1.

Compounds of formula I and their pharmaceutically acceptable acid addition salts, hereinafter referred to as agents of the invention, exhibit valuable pharmacological properties when tested in vitro and in animals, and are therefore useful as pharmaceuticals.

The agents of the invention are inhibitors of aspartic proteases and can be used for the treatment of disorders involving processing by such enzymes. Particularly they inhibit beta-secretase and as such inhibit the generation of beta-amyloid and the subsequent aggregation into oligomers and fibrils.

Test 1 Inhibition of BACE

Recombinant BACE (extracellular domain, expressed in baculovirus and purified using standard methods) at 6 nM concentration is incubated with test compound at various concentrations for 1 hour at room temperature in 100 mM acetate buffer, pH 4.5, containing 0.1% CHAPS. Synthetic peptide substrate Mca-Ser-Glu-Val-Asn-Leu-Asp-Ala-Glu-Phe-Lys(DNP) is added to a final concentration of 3 μM and increase in fluorescence is recorded at excitation of 325 nm and emission at 400 nm in a microplate spectro-fluorimeter for 20 minutes in 1-minute intervals. IC₅₀ values are calculated from percentage of inhibition of BACE-activity as a function of test compound concentration.

Test 2 Inhibition of BACE-2

Recombinant BACE-2 (extracellular domain, expressed in baculovirus and purified using standard methods) at 2.5 nM concentration is incubated with test compound at various concentrations for 1 hour at room temperature in 100 mM acetate buffer, pH 4.5, containing 0.1% CHAPS. Synthetic peptide substrate Mca-Ser-Glu-Val-Asn-Leu-Asp-Ala-Glu-Phe-Lys(DNP) is added to a final concentration of 3 μM and increase in fluorescence is recorded at excitation of 325 nm and emission at 400 nm in a microplate spectro-fluorimeter for 20 minutes in 1-minute intervals. IC₅₀ values are calculated from percentage of inhibition of BACE-2-activity as a function of test compound concentration.

Test 3 Inhibition of Cathepsin D

Recombinant cathepsin D (expressed as procathepsin D in baculovirus, purified using standard methods and activated by incubation in sodium formate buffer pH 3.7) is incubated with test compound at various concentrations for 1 hour at room temperature in 100 mM sodium formate buffer, pH 3.1. Synthetic peptide substrate Mca-Gly-Lys-Pro-Ile-Leu-Phe-Phe-Arg-Leu-Lys(DNP)-D-Arg-NH₂ is added to a final concentration of 2 μM and increase in fluorescence is recorded at excitation of 325 nm and emission at 400 nm in a microplate spectro-fluorimeter for 20 minutes in 1-minute intervals. IC₅₀ values are calculated from percentage of inhibition of cathepsin D-activity as a function of test compound concentration. In the described assay, the agents of the present invention show IC₅₀ values between about 0.2 and 5 μM, in particular between 0.2 and 2 μM.

Test 4 Inhibition of Cellular Release of Amyloid Peptide 1-40

Chinese hamster ovary cells are transfected with the gene for amyloid precursor protein. Cells are plated at a density of 8000 cells/well in a 96-well microtiter plate and cultivated for 24 hours in DMEM cell culture medium containing 10% FCS. Test compound is added to the cells at various concentrations, and cells are cultivated for 24 hours in presence of test compound. Supernatants are collected, and concentration of amyloid peptide 1-40 is determined using sandwich ELISA. Potency of the compound is calculated from the percentage of inhibition of amyloid peptide release as a function of test compound concentration.

Test 5 Cytotoxicity

Cytotoxicity of test compound is determined using Chinese hamster ovary cells transfected with the gene for amyloid precursor protein. Cells are plated at a density of 8000 cells/well in a 96-well microtiter plate and cultivated for 24 hours in DMEM cell culture medium containing 10% FCS. Test compound is added to the cells at various concentrations, and cells are cultivated for 24 hours in presence of test compound. After removing the supernatant, the percentage of living and dead cells is determined using a colorimetric readout (MTS-method), which measures mitochondrial dehydrogenase enzymes in living cells.

The agents of the invention are therefore useful e.g. for the treatment and/or prevention of neurological and vascular disorders related to beta-amyloid generation and/or aggregation such as neurodegenerative diseases like Alzheimer's disease, Down's Syndrome, memory and cognitive impairment, dementia, amyloid neuropathies, brain inflammation, nerve and brain trauma, vascular amyloidosis, or cerebral haemorrhage with amyloidosis.

Some of the agents of the invention also inhibit BACE2 (beta-site APP-cleaving enzyme 2) or Cathepsin D, close homologues of beta-secretase. Due to the correlation of BACE2 and CathD expression with a more tumorigenic and metastatic potential of tumor cells, such inhibitors are useful for the suppression of the metastasis process associated with tumor cells.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 1 to about 50 mg/kg animal body weight. In larger mammals, for example humans, an indicated daily dosage is in the range from about 10 to about 2000, preferably from about 10 to about 200 mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a pharmaceutical, e.g. for the treatment of neurological and vascular disorders related to beta-amyloid generation and/or aggregation.

The present invention furthermore provides a pharmaceutical composition comprising an agent of the invention in association with at least one pharmaceutical carrier or diluent. Such compositions may be manufactured in conventional manner. Unit dosage forms contain, for example, from about 1 to about 1000, preferably from about 1 to about 500 mg of an agent of the invention.

The agents of the invention can be administered alone or in combination with other pharmaceutical agents effective, in the treatment of conditions mentioned above. The other pharmaceutical agents can be selected especially from donepezil hydrochloride, e.g., in the form as marketed under the trademark Aricept™, rivastigmine, e.g., in the form as marketed, e.g. under the trademark Exelon™ and galantamine hydrobromide e.g., in the form as marketed, e.g. under the trademark Reminyl™.

The pharmaceutical combination may be in form of a unit dosage form, whereby each unit dosage will comprise a predetermined amount of the two components, in admixture with suitable pharmaceutical carriers or diluents. Alternatively, the combination may be in form of a package containing the two components separately, e.g. a pack or dispenser-device adapted for the concomitant or separate administration of the two active agents, wherein these agents are separately arranged.

Moreover the present invention provides the use of an agent of the invention, for the manufacture of a medicament for the treatment of any neurological and vascular disorders related to beta-amyloid generation and/or aggregation.

In still a further aspect the present invention provides a method for the treatment of any neurological and vascular disorders related to beta-amyloid generation and/or aggregation, in a subject in need of such treatment, which comprises administering to such subject a therapeutically effective amount of an agent of the invention.

The following examples illustrate the invention.

### GENERAL ABBREVIATIONS

BOC tert-butoxycarbonyl

BOP benotriazol-1-yloxytris(dimethylamino)-phosphonium hexafluorophosphate

DMPU N,N′-dimethylpropyleneurea

EDCI 1-ethyl-3-[3-(dimethylamino)propyl]-carbodiimide hydrochloride

EtOAc ethylacetate

h hours

HOBt hydroxybenztriazole

HPLC high pressure liquid chromatography

min minutes

MS mass spectroscopy

rt room temperature

TFA trifluoroacetic acid

THF tetrahydrofuran

### EXAMPLE 1

### (2R,4S)—N-Butyl-4-hydroxy-2-methyl-4-((10S,13S)-10-methyl-8,11-dioxo-2-oxa-9,12-diaza-bicyclo[13.3.1]nonadeca-1(18),15(19),16-trien-13-yl)-butyramide

### a) [1-Benzenesulfonyl-2-(3-benzyloxy-phenyl)-ethyl]-carbamic acid tert-butyl ester

A suspension of (3-Benzyloxy-phenyl)-acetaldehyde (20.6 g, 91 mmol), tert-butylcarbamate (10.7 g, 91 mmol, 1 eq), sodium benzenesulfinate (18.3 g, 109 mmol, 1.2 eq) and formic acid (5.2 ml, 137 mmol, 1.5 eq) in 155 ml acetonitrile is stirred at 80° C. for 4 h. After cooling to rt the mixture is taken up in EtOAc. The solution is washed with bicarbonate and brine, dried over magnesium sulfate and evaporated. The residue (37.3 g) is used for the next step without further purification.

### b) [(S*)-2-(3-Benzyloxy-phenyl)-1-((S*)-5-oxo-2,5-dihydro-furan-2-yl)-ethyl]-carbamic acid tert-butyl ester

5H-Furan-2-one (11.2 ml, 160 mmol, 2 eq) in THF (60 ml) is added slowly to a solution of lithium diisopropylamide (80 ml commercial 2M solution in THF/heptane/ethylbenzene, 160 mmol, 2 eq) in THF (180 ml) at −78° C. The mixture is stirred for another 20 min at −78° C. before [1-Benzenesulfonyl-2-(4-benzyloxy-phenyl)-ethyl]-carbamic acid tert-butyl ester (37.3 g, 80 mmol) in THF (220 ml) is added at the same temperature. After stirring for another 45 min at −78° C. aqueous bicarbonate solution is added and the reaction mixture is taken up into EtOAc. The organic layer is washed with bicarbonate and brine and dried over magnesium sulfate. Evaporation of the solvent gives a residue that is purified by chromatography on silica using hexan/EtOAc 9/1 to 7/3. The product is recrystallized from ether/hexane to give the product as white crystals.

1H-NMR (400 MHz, CDCl3): 7.45-7.2 (m, 7H), 6.9-6.85 (m, 3H), 6.06 (d, 1H), 5.07 (s, 2H), 4.90 (d, 1H), 4.50 (d, 1H), 4.20 (q, 1H), 3.01 (dd, 1H), 2.91 (dd, 1H), 1.38 (s, 9H).

### c) [(S*)-2-(3Benzyloxy-phenyl)-1-((S*)-5-oxo-tetrahydro-furan-2-yl)-ethyl]-carbamic acid tert-butyl ester

[(S*)-2-(4-Benzyloxy-phenyl)-1-((S*)-5-oxo-2,5-dihydro-furan-2-yl)-ethyl]-carbamic acid tert-butyl ester (11.1 g, 27 mmol) is hydrogenated (1 atm H₂) at rt in THF (550 ml) with Pt/C as catalyst (5% Engelhard 4709, 2.3 g) during 1 h. The catalyst is filtered off and the filtrate is evaporated. Purification by chromatography on silica (Flashmaster, hexane to hexane/EtOAc 55/45 over 40 min) gives the product as yellowish oil.

1H-NMR (400 MHz, CDCl3): 7.45-7.2 (m, 6H), 6.9-6.8 (m, 3H), 5.06 (s, 2H), 4.61 (d, 1H), 4.44 (t, 1H), 4.00 (q, 1H), 2.95 (dd, 1H), 2.85 (dd, 1H), 2.6-2.45 (m, 2H), 2.15-2.1 (m, 2H), 1.42 (s, 9H).

### d) [(S*)-2-(3-Benzyloxy-phenyl)-1-((2S*,4R*)-4-methyl-5-oxo-tetrahydro-furan-2-yl)-ethyl]-carbamic acid tert-butyl ester

To a solution of [(S*)-2-(4-Benzyloxy-phenyl)-1-((S*)-5-oxo-2,5-dihydro-furan-2-yl)-ethyl]-carbamic acid tert-butyl ester (11.4 g, 27.7 mmol) in THF (35 ml) and DMPU (5 ml, 42 mmol, 1.5 eq) at −78° C. is added dropwise lithium-bis-(trimethylsilyl)-amide (55 ml 1 M solution in THF, 55 mmol, 2 eq). After stirring at −78° C. for another 45 min methyliodide is added dropwise and the mixture is stirred another 3 h at −78° C. Propionic acid (10.3 ml, 138 mmol, 5 eq) is added followed by water (10 ml). After warming up to 0° C. a 10% solution of citric acid (72 ml) is added. The reaction mixture is extracted with EtOAc. The organic layer is washed with bicarbonate, 0.1N sodium sulfite and brine, dried over magnesium sulfate and evaporated. Purification by chromatography on silica (hexane/EtOAc 9/1 to 4/1) followed by recrystallization from ether/hexane gives white crystals.

MS (LC/MS): 448 (M+Na)

1H-NMR (400 MHz, CDCl3): 7.45-7.2 (m, 6H), 6.9-6.8 (m, 3H), 5.05 (s, 2H), 4.53 (d, 1H), 4.45 (t, 1H), 4.00 (q, 1H), 2.93-2.85 (m, 2H), 2.74-2.68 (m, 1H), 2.41-2.34 (m, 1H), 1.89-1.82 (m, 1H), 1.41 (s, 9H), 1.26 (d, 3H).

### e) [(1S*,2S*,4R*)-1-(3-Benzyloxy-benzyl)-4-butylcarbamoyl-2-hydroxy-pentyl]-carbamic acid tert-butyl ester

[(S*)-2-(3-Benzyloxy-phenyl)-1-((2S*,4R*)-4-methyl-5-oxo-tetrahydro-furan-2-yl)-ethyl]-carbamic acid tert-butyl ester (4.0 g, 9.4 mmol) is dissolved in butylamine (200 ml) and stirred for 18 h in a heating bath of 90° C. The butylamine is evaporated and the residue is recrystallized from dichloromethane/ether/hexane to give white crystals.

MS (LC/MS): 521 (M+Na)

1H-NMR (400 MHz, CDCl3): 7.45-7.15 (m, 6H), 6.9-6.8 (m, 3H), 5.91 (s, 1H), 5.04 (s, 2H), 4.89 (d, 1H), 3.7-3.6 (m, 2H), 3.3-3.1 (m, 2H), 2.9-2.85 (m, 2H), 2.6-2.5 (m, 1H), 1.75-1.6 (m, 2H), 1.5-1.25 (m, 4H), 1.41 (s, 9H), 1.12 (d, 3H), 0.92 (t, 3H).

### f) {(S)-1-[(1S*,2S*,4R*)-1-(3-Benzyloxy-benzyl)-4-butylcarbamoyl-2-hydroxy-pentylcarbamoyl]-ethyl}-carbamic acid tert-butyl ester

[(1S*,2S*,4R*)-1-(3-Benzyloxy-benzyl)-4-butylcarbamoyl-2-hydroxy-pentyl]-carbamic acid tert-butyl ester (4.4 g, 8.8 mmol) is dissolved in trifluoroacetic acid (95 ml) and stirred at rt during 1.25 h. The solvent is evaporated and the residue is a few times redissolved in ether and dried again to give a white foam (3.92 g, 9.0 mmol). The foam is dissolved in dichloromethane (210 ml) and BOC-L-alanine (1.05 g, 10.8 mmol, 1.2 eq), EDCI (2.59 g, 13.5 mmol, 1.5 eq), HOBt (1.46 g, 10.8 mmol, 1.2 eq) and triethylamine (3.76 g, 27 mmol, 3 eq) are added. The reaction is stirred at rt (with an argon balloon) for 18 h. The mixture is extracted with EtOAc and the organic layer is washed with 0.5N HCl, brine, bicarbonate and brine again, and dried over magnesium sulfate. Evaporation of the solvent and recrystallization of the residue from dichloromethane/ether/hexane gives the product as an about 1/1 mixture of diastereomers.

MS (LC/MS): 593 (M+Na)

1H-NMR (400 MHz, CDCl3): 7.45-7.15 (m, 6H), 6.9-6.8 (m, 3H), 6.47/6.30 (d, 1H), 5.90 (s, 1H), 5.08 (s, 2H), 5.0-4.85 (m, 1H), 4.1-3.98 (m, 2H), 3.77-3.70 (m, 1H), 3.3-3.15 (m, 2H), 2.95-2.85 (m, 2H), 2.6-2.5 (m, 1H), 1.8-1.55 (m, 3H), 1.5-1.4 (m, 10H), 1.4-1.3 (m, 2H), 1.26/1.20 (d, 3H), 1.12-1.08 (m, 3H), 0.95-0.9 (m, 3H).

### g) {(S)-1-[(1S*,2S*,4R*)-4-Butylcarbamoyl-2-hydroxy-1-(3-hydroxy-benzyl)-pentylcarbamoyl]-ethyl}-carbamic acid tert-butyl ester

{(S)-1-[(1S*,2S*,4R*)-1-(3-Benzyloxy-benzyl)-4-butylcarbamoyl-2-hydroxy-pentylcarbamoyl]-ethyl}-carbamic acid tert-butyl ester (4.44 g, 7.8 mmol) is hydrogenated at rt (1 atm H₂) in ethanol (240 ml) with Pd/C (10% Engelhard 4505, 0.69 g) for 4 h. The catalyst is filtered off and the filtrate is evaporated to give a white foam (about 1/1 mixture of diastereomers).

MS (LC/MS): 502 (M+Na)

1H-NMR (400 MHz, CDCl3): 7.14 (t, 1H), 6.78-6.7 (m, 3H), 6.65/6.55 (d, 1H), 6.25/6.08 (s, 1H), 5.26/5.08 (d, 1H), 4.15-3.95 (m, 2H), 3.75-3.7 (m, 1H), 3.3-3.15 (m, 2H), 2.93-2.8 (m, 2H), 2.65-2.52 (m, 1H), 2.0-1.5 (m, 5H), 1.5-1.4 (m, 10H), 1.4-1.3 (m, 2H), 1.30/1.25 (d, 3H), 1.15-1.10 (m, 3H), 0.94 (t, 3H).

### h) 6-{3-[(2S*,3S*,5R*)-2-((S)-2-tert-Butoxycarbonylamino-propionylamino)-5-butylcarbamoyl-3-hydroxy-hexyl]-phenoxy}-hexanoic acid tert-butyl ester

{(S)-1-[(1S*,2S*,4R*)-4-Butylcarbamoyl-2-hydroxy-1-(3-hydroxy-benzyl)-pentylcarbamoyl]-ethyl}-carbamic acid tert-butyl ester (1.20 g, 2.5 mmol) is dissolved in acetone (250 ml) and treated with 6-Bromo-hexanoic acid tert-butyl ester (CAS 65868-63-5, 0.94 g, 3.8 mmol, 1.5 eq), water free potassium carbonate (1.04 g, 7.5 mmol, 3 eq) and potassium iodide (0.42 g, 2.5 mmol, 1 eq). The mixture is heated at reflux temperature (bath temperature 75° C.) for 5 days. The reaction mixture is diluted with EtOAc, washed with brine, dried over magnesium sulfate and evaporated. The crude product is purified by chromatography on silica (flashmaster, dichloromethane to dichloromethane/MeOH 90/10; mixed fractions are collected and resubmitted to chromatography). After a total of 5 chromatographies and recrystallization from ether/hexane white crystals are obtained (about 1/1 mixture of diastereomers).

MS (LC/MS): 672 (M+Na)

1H-NMR (400 MHz, C2D2Cl4): 7.25 (t, 1H), 6.88-6.82 (m, 3H), 6.32/6.20 (d, 1H), 5.33/5.17 (s, 1H), 4.43/4.33 (d, 1H), 4.15-4.03 (m, 4H), 3.82 (s, 1H), 3.45-3.2 (m, 3H), 3.03-2.90 (m, 2H), 2.60-2.53 (m, 1H), 2.33 (t, 2H), 1.90-1.80 (m, 2H), 1.80-1.65 (m, 3H), 1.65-1.4 (m, 25H), 1.35/1.32 (d, 3H), 1.19 (d, 3H), 1.03 (t, 3H).

### i) (S)-1-{(1S,2S,4R)-4-Butylcarbamoyl-1-[3-(5-carboxy-pentyloxy)-benzyl]-2-hydroxy-pentylcarbamoyl}-ethyl-ammonium trifluoroacetate

6-{3-[(2S*,3S*,5R*)-2-((S)-2-tert-Butoxycarbonylamino-propionylamino)-5-butylcarbamoyl-3-hydroxy-hexyl]-phenoxy}-hexanoic acid tert-butyl ester (1.25 g, 1.9 mmol) is dissolved in trifluoroacetic acid (40 ml) and water (4.5 ml) at 0° C. and stirred for 30 min. The reaction mixture is diluted with 380 ml cold water and the solvents are evaporated at rt. The two enantiomerically pure diastereomers are separated by preparative HPLC (Nucleosil 100-5 C18, water/acetonitrile 90/10 to acetonitrile). The first diastereomer is collected in about 80% purity and used directly for the next step, MS (LC/MS): 516 (M+Na).

### j) (2R,4S)-N-Butyl-4-hydroxy-2-methyl-4-((10S,13S)-10-methyl-8,11-dioxo-2-oxa-9,12-diaza-bicyclo[13.3.1]nonadeca-1(18),15(19),16-trien-13-yl)-butyramide

(S)-1-{(1S,2S,4R)-4-Butylcarbamoyl-1-[3-(5-carboxy-pentyloxy)-benzyl]-2-hydroxy-pentylcarbamoyl}-ethyl-ammonium trifluoroacetate (0.26 g, 0.43 mmol) is dissolved in DMF (60 ml) and BOP (0.28 g, 0.64 mmol, 1.5 eq) and diisopropylethylamine (0.47 ml, 2.8 mmol, 6.5 eq) are added. The reaction mixture is stirred over night at rt. The solvent is evaporated at rt and the residue is taken into EtOAc. Washing with 0.5N HCl, brine, bicarbonate and brine again, drying over magnesium sulfate and evaporation of the solvent gives the crude product. This is purified by preparative HPLC (Nucleosil 100-5 C18, water/acetonitrile 90/10 to acetonitrile) to give the title compound as white powder after liophilisation.

MS (LC/MS): 498 (M+Na)

1H-NMR (400 MHz, CD3OD): 7.15 (t, 1H), 6.8-6.7 (m, 3H), 4.53 (q, 1H), 4.15-4.05 (m, 2H), 4.05 (d, 1H), 3.60 (dt, 1H), 3.18 (t, 2H), 2.86 (d, 1H), 2.73-2.6 (m, 2H), 2.27-2.22 (m, 1H), 2.16-2.10 (m, 1H), 1.84-1.65 (m, 4H), 1.55-1.3 (m, 8H), 1.27 (d, 3H), 1.17 (d, 3H), 0.95 (t, 3H).

### EXAMPLE 2

### (2R*,4S*)-N-Butyl-4-hydroxy-2-methyl-4-((9S,12S*)-9-methyl-7,10-dioxo-2-oxa-8,11-diaza-bicyclo[12.3.1]octadeca-1(18),14,16-trien-12-yl)-butyramide

### a) 5-{3-[(2S*,3S*,5R*)-2-((S)-2-tert-Butoxycarbonylamino-propionylamino)-5-butylcarbamoyl-3-hydroxy-hexyl]-phenoxy}-pentanoic acid tert-butyl ester

{(S)-1-(1S*,2S*,4R*)-4-Butylcarbamoyl-2-hydroxy-1-(3-hydroxy-benzyl)-pentylcarbamoyl]-ethyl}-carbamic acid tert-butyl ester (see example 1 g, 1.30 g, 2.7 mmol) is dissolved in acetone (250 ml) and treated with 5-bromo-pentanoic acid tert-butyl ester (CAS 88987-42-2, 0.96 g, 4.1 mmol, 1.5 eq), water free potassium carbonate (1.12 g, 8.1 mmol, 3 eq) and potassium iodide (0.45 g, 2.7 mmol, 1 eq). The mixture is heated at reflux temperature (bath temperature 75° C.) for 7 days. The reaction mixture is diluted with EtOAc, washed with brine, dried over magnesium sulfate and evaporated. The crude product is purified by chromatography on silica (flashmaster, dichloromethane to dichloromethane/MeOH 90/10; mixed fractions are collected and resubmitted to chromatography). After a total of 4 chromatographies a white foam is obtained (about 1/1 mixture of diastereomers).

MS (LC/MS): 658 (M+Na)

1H-NMR (400 MHz, CDCl3): 7.17 (t, 1H), 6.79-6.70 (m, 3H), 6.52 (d, 1H), 6.35 (d, 1H), 5.94 (t, 1H), 5.1-4.8 (m, 1H), 4.1-3.93 (m, 4H), 3.75-3.7 (m, 1H), 3.3-3.1 (m, 2H), 2.94-2.84 (m, 2H), 2.60-2.50 (m, 1H), 2.29 (t, 2H), 1.85-1.55 (m, 7H), 1.55-1.4 (m, 18H), 1.4-1.18 (m, 6H), 1.12-1.08 (m, 3H), 0.92 (t, 3H).

### b) (S)-1-{(1S*,2S*,4R*)-4-Butylcarbamoyl-1-[3-(4-carboxy-butoxy)-benzyl]-2-hydroxy-pentylcarbamoyl}-ethyl-ammonium trifluoroacetate

5-{3-[(2S*,3S*,5R*)-2-((S)-2-tert-Butoxycarbonylamino-propionylamino)-5-butylcarbamoyl-3-hydroxy-hexyl]-phenoxy}-pentanoic acid tert-butyl ester (1.46 g, 2.3 mmol) is dissolved in trifluoroacetic acid (49.5 ml) and water (5.5 ml) at 0° C. and stirred for 30 min. The solvents are evaporated at rt. The residue is dissolved in dichloromethane and dried over sodium sulfate. Evaporation of the solvent yields a viscous oil that is used directly for the next step.

### c) (2R*,4S*)-N-Butyl-4-hydroxy-2-methyl-4-((9S,12S*)-9-methyl-7,10-dioxo-2-oxa-8,11-diaza-bicyclo[12.3.1]octadeca-1(18),14,16-trien-12-yl)-butyramide

(S)-1-{(1S*,2S*,4R*)-4-Butylcarbamoyl-1-[3-(4-carboxy-butoxy)-benzyl]-2-hydroxy-pentylcarbamoyl}-ethyl-ammonium trifluoroacetate (0.90 g, 1.5 mmol) is dissolved in DMF (210 ml) and BOP (1.0 g, 2.3 mmol, 1.5 eq) and diisopropylethylamine (1.69 ml, 9.9 mmol, 6.5 eq) are added. The reaction mixture is stirred over night at rt. The solvent is evaporated at rt and the residue is taken into EtOAc. Washing with 0.5N HCl, brine, bicarbonate and brine again, drying over magnesium sulfate and evaporation of the solvent gives the crude product. This is purified by chromatography on silica (flashmaster, dichloromethane to dichloromethane/MeOH 90/10) followed by preparative HPLC (Nucleosil 100-5 C18, water/acetonitrile 90/10 to acetonitrile) to give the title compound as white powder after liophilisation (12 mg, mixture of diastereomers).

MS (LC/MS): 484 (M+Na)

1H-NMR (400 MHz, DMSO): 8.04/7.85 (d, 1H), 7.93/7.42 (d, 1H), 7.60 (t, 1H), 7.10 (t, 1H), 6.8-6.6 (m, 3H), 4.95/4.70 (d, 1H), 4.2-4.0 (m, 2H), 3.95-3.35 (m, 3H), 3.05-2.9 (m, 2H), 2.85-2.55 (m, 2H), 2.4-2.3 (m, 2H), 2.25-2.05 (m, 1H), 1.75-1.45 (m, 5H), 1.45-0.9 (m, 10H), 0.87 (t, 3H).

### EXAMPLE 3

### (2R*,4S*)-N-Butyl-4-hydroxy-2-methyl-4-((8S,11S*)-8-methyl-6,9-dioxo-2-oxa-7,10-diaza-bicyclo[11.3.1]heptadeca-1(17),13,15-trien-11-yl)-butyramide

### a) 4-{3-[(2S*,3S*,5R*)-2-((S)-2-tert-Butoxycarbonylamino-propionylamino)-5-butylcarbamoyl-3-hydroxy-hexyl]-phenoxy}-butyric acid tert-butyl ester

{(S)-1-[(1S*,2S*,4R*)-4-Butylcarbamoyl-2-hydroxy-1-(3-hydroxy-benzyl)-pentylcarbamoyl]-ethyl}-carbamic acid tert-butyl ester (see example 1 g, 1.30 g, 2.7 mmol) is dissolved in acetone (250 ml) and treated with 5-Bromo-pentanoic acid tert-butyl ester (CAS 88987-42-2, 0.96 g, 4.1 mmol, 1.5 eq), water free potassium carbonate (1.12 g, 8.1 mmol, 3 eq) and potassium iodide (0.45 g, 2.7 mmol, 1 eq). The mixture is heated at reflux temperature (bath temperature 75° C.) for 7 days. The reaction mixture is diluted with EtOAc, washed with brine, dried over magnesium sulfate and evaporated. The crude product is purified by chromatography on silica (flashmaster, dichloromethane to dichloromethane/MeOH 90/10; mixed fractions are collected and resubmitted to chromatography). A white foam is obtained (about 1/1 mixture of diastereomers).

MS (LC/MS): 644 (M+Na)

### b) (S)-1-{(1S*,2S*,4R*)-4-Butylcarbamoyl-1-[3-(3-carboxy-propoxy)-benzyl]-2-hydroxy-pentylcarbamoyl}-ethyl-ammonium trifluoroacetate

4-{3-[(2S*,3S*,5R*)-2-((S)-2-tert-Butoxycarbonylamino-propionylamino)-5-butylcarbamoyl-3-hydroxy-hexyl]-phenoxy}-butyric acid tert-butyl ester (1.45 g, 2.3 mmol) is dissolved in trifluoroacetic acid (49.5 ml) and water (5.5 ml) at 0° C. and stirred for 30 min. The solvents are evaporated at rt. The residue is dissolved in dichloromethane and dried over sodium sulfate. Evaporation of the solvent yields a viscous oil that is used directly for the next step.

MS (LC/MS): 488 (M+Na)

### c) (2R*,4S*)-N-Butyl-4-hydroxy-2-methyl-4-((8S,11S*)-8-methyl-6,9-dioxo-2-oxa-7,10-diaza-bicyclo[11.3.1]octadeca-1(17),13,15-trien-11-yl)-butyramide

(S)-1-{(1S*,2S*,4R*)-4-Butylcarbamoyl-1-[3-(3-carboxy-propoxy)-benzyl]-2-hydroxy-pentylcarbamoyl}-ethyl-ammonium trifluoroacetate (0.77 g, 1.3 mmol) is dissolved in DMF (180 ml) and BOP (0.88 g, 2.0 mmol, 1.5 eq) and diisopropylethylamine (1.47 ml, 8.6 mmol, 6.5 eq) are added. The reaction mixture is stirred over night at rt. The solvent is evaporated at rt and the residue is taken into EtOAc. Washing with 0.5N HCl, brine, bicarbonate and brine again, drying over magnesium sulfate and evaporation of the solvent gives the crude product. This is purified by chromatography on silica (flashmaster, dichloromethane to dichloromethane/MeOH 90/10) followed by preparative HPLC (Nucleosil 100-5 C18, water/acetonitrile 90/10 to acetonitrile) to give the title compound as white powder after liophilisation (mixture of diastereomers).

MS (LC/MS): 470 (M+Na)

1H-NMR (400 MHz, DMSO): 7.95-7.55 (m, 2H), 7.10-7.00 (m, 1H), 6.8-6.6 (m, 3H), 4.7-4.5 (m, 1H), 4.3-4.1 (m, 1H), 3.95-3.85 (m, 1H), 3.7-3.6 (m, 1H), 3.4-3.3 (m, 2H), 3.05-2.9 (m, 2H), 2.7-2.5 (m, 5H), 2.35-2.15 (m, 1H), 2.0-1.65 (m, 3H), 1.45-1.15 (m, 6H), 1.05-0.95 (m, 4H), 0.87 (t, 3H).

### EXAMPLE 4

### (2R*,4S*)-N-Butyl-4-hydroxy-2-methyl-4-((11S*,14S*)-11-methyl-9,9,12-trioxo-2-oxa-9lambda*6*-thia-13-aza-bicyclo[14.3.1]icosa-1(20),16,18-trien-14-yl)-butyramide

### a) [(1S*,2S*,4R*)-4-Butylcarbamoyl-2-hydroxy-1-(3-hydroxy-benzyl)-pentyl]-carbamic acid tert-butyl ester

[(1S*,2S*,4R*)-1-(3-Benzyloxy-benzyl)-4-butylcarbamoyl-2-hydroxy-pentyl]-carbamic acid tert-butyl ester (see example 1e, 0.24 g, 0.48 mmol) is hydrogenated at rt (1 atm H₂) in ethanol (30 ml) with Pd/C (10% Engelhard 4505, 0.060 g) for 2 h. The catalyst is filtered off and the filtrate is evaporated. The crude product is purified by chromatography on silica (flashmaster, dichloromethane to dichloromethane/MeOH 85/15) to give the product as a white foam; MS (LC/MS): 431 (M+Na).

### b) [(1S*,2S*,4R*)-1-(3-Allyloxy-benzyl)-4-butylcarbamoyl-2-hydroxy-pentyl]-carbamic acid tert-butyl ester

[(1S*,2S*,4R*)-4-Butylcarbamoyl-2-hydroxy-1-(3-hydroxy-benzyl)-pentyl]-carbamic acid tert-butyl ester (0.60 g, 1.47 mmol), water free potassium carbonate (0.61 g, 4.4 mmol, 3 eq), potassium iodide (0.244 g, 1.47 mmol, 1 eq) and 3-bromo-propene (0.186 ml, 2.2 mmol, 1.5 eq) are dissolved in dry acetone (150 ml) and stirred at reflux temperature (bath temperature 75° C.) during 3 days. The reaction mixture is diluted with EtOAc, washed with brine, dried over magnesium sulfate and the solvents are evaporated. Purification by chromatography on silica (flashmaster, dichloromethane to dichloromethane/MeOH 90/10) followed by recrystallization from ether/hexane gives the product as white solid.

MS (LC/MS): 471 (M+Na)

### c) (2R-,4S*,5S*)-6-(3-Allyloxy-phenyl)-4-hydroxy-2-methyl-5-((S*)-2-(pent-4-ene-1-sulfonyl)-propionylamino]-hexanoic acid butylamide

[(1S*,2S*,4R*)-1-(3-Allyloxy-benzyl)-4-butylcarbamoyl-2-hydroxy-pentyl]-carbamic acid tert-butyl ester (0.26 g, 0.58 mmol) is dissolved in 4N hydrochloric acid in dioxane and stirred at rt for 1.5 h. The solvent is evaporated and the residue is dried at low pressure. The residue is redissolved in dry dichloromethane (20 ml). Racemic 2-Methyl-3-(pent-4-ene-1-sulfonyl)-propionic acid (see intermediate 3, 0.165 g, 0.75 mmol, 1.2 eq), EDCI (0.179 g, 0.94 mmol, 1.5 eq), HOBt (0.115 g, 0.75 mmol, 1.2 eq) and triethylamine (0.26 ml, 1.87 mmol, 3 eq) are added. The reaction mixture is stirred under argon at rt for 18 h. The mixture is diluted with EtOAc, washed with 0.5N aqueous HCl, brine, sodium bicarbonate solution and again with brine. Drying over magnesium sulfate and evaporation of the solvents gives the crude product (mixture of two racemic diastereomers). The diastereomeres are separated by chromatography on silica (flashmaster, EtOAc/hexanes 1/1 to EtOAc), mixed fractions are collected and separated again on a preparative TLC plate to give the desired racemic diastereomer (eluted as second compound, Rf=0.47 in EtOAc).

MS (LC/MS): 573 (M+Na)

### d) (2R*,4S*)-N-Butyl-4-hydroxy-2-methyl-4-((11S*,14S*)11-methyl-9,9,12-trioxo-2-oxa-9lambda*6*-thia-13-aza-bicyclo[14.3.1]icosa-1(20),16,18-trien-14-yl)-butyramide

(2R*,4S*,5S*)-6-(3-Allyloxy-phenyl)-4-hydroxy-2-methyl-5-[(S*)-2-(pent-4-ene-1-sulfonyl)-propionylamino]-hexanoic acid butylamide (0.115 g, 0.21 mmol) is dissolved in dry dichloromethane (220 ml) under argon and benzylidene(1,3-dimesitylimidazolidin-2-ylidene)(tricyclohexylphosphine)ruthenium dichloride (CAS 246047-72-3) (9 mg, 5 mol %) is added. The reaction is stirred at reflux temperature (bath temperature 60° C.) for 2.5 h. The solvent is evaporated and the residue is purified by chromatography on silica (flashmaster, EtOAc/hexanes 1/1 to EtOAc). The resulting off-white solid is hydrogenated with Pd/C (10% Engelhard 4505, 0.020 g) in methanol (3 ml) at rt at 1 atm hydrogen during 3 h. After filtering through glass wool the solvent is evaporated and the crude product is purified on a preparative TLC plate (dichloromethane/MeOH 95/5). Recrystallization from dichloromethane/ether/hexane gives the product.

MS (LC/MS): 547 (M+Na)

1H-NMR (400 MHz, CDCl3): 7.23 (t, 1H), 6.84-6.76 (m, 3H), 6.15 (d, 1H), 5.88 (t, 1H), 4.31 (t, 1H), 4.18-4.10 (m, 2H), 3.90 (d, 1H), 3.33-3.18 (m, 3H), 3.01-2.84 (m, 3H), 2.71-2.54 (m, 4H), 1.85-1.60 (m, 7H), 1.60-1.45 (m, 6H), 1.45-1.32 (m, 2H), 1.31 (d, 3H), 1.28 (d, 3H), 0.96 (t, 3H).

### EXAMPLE 5

### (2R,4S)—N-Butyl-4-hydroxy-2-methyl-4-((9S,12S)-9-methyl-7,7,10-trioxo-2-oxa-7lambda*6*-thia-11-aza-bicyclo[12.3.1]octadeca-1(18),14,16-trien-12-yl)-butyramide

### a) (2R*,4S*,5S*)-6-(3-Allyloxy-phenyl)-4-hydroxy-2-methyl-5-[(S)-2-methyl-3-(prop-2-ene-1-sulfonyl)-propionylamino]-hexanoic acid butylamide

[(1S*,2S*,4R*)-1-(3-Allyloxy-benzyl)-4-butylcarbamoyl-2-hydroxy-pentyl]-carbamic acid tert-butyl ester (see example 4b, 0.275 g, 0.61 mmol) is dissolved in 4N hydrochloric acid in dioxane and stirred at rt for 1.5 h. The solvent is evaporated and the residue is dried at low pressure. The residue is redissolved in dry dichloromethane (20 ml). (S)-2-Methyl-3-(prop-2-ene-1-sulfonyl)-propionic acid (see intermediate 2, 0.138 g, 0.72 mmol, 1.2 eq), EDCI (0.172 g, 0.90 mmol, 1.5 eq), HOBt (0.110 g, 0.72 mmol, 1.2 eq) and triethylamine (0.25 ml, 1.8 mmol, 3 eq) are added. The reaction mixture is stirred under argon at rt for 18 h. The mixture is diluted with EtOAc, washed with 0.5N aqueous HCl, brine, sodium bicarbonate solution and again with brine. Drying over magnesium sulfate, evaporation of the solvents and purification by chromatography on silica (flashmaster, dichloromethane to dichloromethane/MeOH 95/5) gives the product as a 1:1 mixture of two enantiomerically pure diastereomers.

MS (LC/MS): 545 (M+Na)

1H-NMR (400 MHz, CDCl3): 7.23-7.18 (m, 1H), 6.87-6.77 (m, 3H), 6.25-5.80 (m, 5H), 5.55-5.42 (m, 3H), 5.31 (d, 1H), 4.55 (d, 2H), 4.4-4.0 (m, 3H), 3.82-3.42 (m, 4H), 3.35-3.17 (m, 2H), 2.97-2.75 (m, 4H), 2.60-2.53 (m, 1H), 1.83-1.60,(m, 3H), 1.53-1.46 (m, 2H), 1.41-1.29 (m, 2H), 1.32/1.17 (d, 3H), 1.13/0.95 (t, 3H).

### b) (2R,4S)—N-Butyl-4-hydroxy-2-methyl-4-((9S,12S)-9-methyl-7,7,10-trioxo-2-oxa-7lambda*6*-thia-11-aza-bicyclo[12.3.1]octadeca-1(18),14,16-trien-12-yl)-butyramide

(2R*,4S*,5S*)-6-(3-Allyloxy-phenyl)-4-hydroxy-2-methyl-5-[(S)-2-methyl-3-(prop-2-ene-1-sulfonyl)-propionylamino]-hexanoic acid butylamide (0.230 g, 0.44 mmol) is dissolved in dry dichloromethane (440 ml) under argon and benzylidene(1,3-dimesitylimidazolidin-2-ylidene)(tricyclohexylphosphine)ruthenium dichloride (CAS 246047-72-3) (19 mg, 5 mol %) is added. The reaction is stirred at reflux temperature (bath temperature 60° C.) for 3 h. The solvent is evaporated and the residue is purified by chromatography on silica (flashmaster, EtOAc/hexanes 1/1 to EtOAc) followed by a second chromatography on silica (flashmaster, dichloromethane to dichloromethane/MeOH 90/10). The resulting solid is recrystallized from dichloromethane/ether/hexane and then hydrogenated with Pd/C (10% Engelhard 4505, 0.035 g) in methanol (5 ml) at rt at 1 atm hydrogen during 3 h. After filtering through glass wool the solvent is evaporated and the two diastereomers are separated by preparative TLC (dichloromethane/MeOH 95/5). Recrystallization of the more polar diastereomer from dichloromethane/ether/hexane gives the product; MS (LC/MS): 519 (M+Na).

1H-NMR (400 MHz, C2D2Cl4): 7.29 (t, 1H), 6.9-6.8 (m, 3H), 5.70 (d, 1H), 5.66 (t, 1H), 4.35-4.18 (m, 3H), 3.95-3.85 (m, 1H), 3.64 (dd, 1H), 3.30 (q, 2H), 3.05-2.8 (m, 5H), 2.68-2.57 (m, 2H), 2.1-1.65 (m, 6H), 1.6-1.4 (m, 4H), 1.37 (d, 3H), 1.30 (d, 3H), 1.02 (t, 3H).

### EXAMPLE 6

### (2R,4S)—N-Butyl-4-hydroxy-2-methyl-4-((10S,13S)-10-methyl-8,8,11-trioxo-2-oxa-8lambda*6*-thia-12-aza-bicyclo[13.3.1]nonadeca-1(19),15,17-trien-13-yl)-butyramide

### a) (2R,4S,5S)-6-(3-Allyloxy-phenyl)-5-[(S)-3-(but-3-ene-1-sulfonyl)-2-methyl-propionylamino]-4-hydroxy-2-methyl-hexanoic acid butylamide

The enantiomers of [(S*)-2-(3-Benzyloxy-phenyl)-1-((2S*,4R*)-4-methyl-5-oxo-tetrahydro-furan-2-yl)-ethyl]-carbamic acid tert-butyl ester (example 1d) are separated by preparative chiral HPLC to give [(S)-2-(3-benzyloxy-phenyl)-1-((2S,4R)-4-methyl-5-oxo-tetrahydro-furan-2-yl)-ethyl]-carbamic acid tert-butyl ester (α_{D}=+10°, c=1 in CHCl3). Derivatization according to example 4a-4b gives [(1S,2S,4R)-1-(3-allyloxy-benzyl)-4-butylcarbamoyl-2-hydroxy-pentyl]-carbamic acid tert-butyl ester.

[(1S,2S,4R)-1-(3-allyloxy-benzyl)-4-butylcarbamoyl-2-hydroxy-pentyl]-carbamic acid tert-butyl ester (0.29 g, 0.58 mmol) is dissolved in 4N hydrochloric acid in dioxane and stirred at rt for 1.5 h. The solvent is evaporated and the residue is dried at low pressure. The residue is redissolved in dry dichloromethane (20 ml). (S)-3-(But-3-ene-1-sulfonyl)-2-methyl-propionic acid (see intermediate 1, 0.158 g, 0.76 mmol, 1.2 eq), EDCI (0.183 g, 0.96 mmol, 1.5 eq), HOBt (0.117 g, 0.76 mmol, 1.2 eq) and triethylamine (0.27 ml, 1.90 mmol, 3 eq) are added. The reaction mixture is stirred under argon at rt for 18 h. The mixture is diluted with EtOAc, washed with 0.5N aqueous HCl, brine, sodium bicarbonate solution and again with brine. Drying over magnesium sulfate and evaporation of the solvents gives the crude product that is purified by chromatography on silica (flashmaster, EtOAc/hexanes 1/1 to EtOAc). Recrystallization from dichloromethane/ether/hexane gives the product.

MS (LC/MS): 559 (M+Na)

### b) (2R,4S)—N-Butyl-4-hydroxy-2-methyl-4-((10S,13S)-10-methyl-8,8,11-trioxo-2-oxa-8lambda*6*-thia-12-aza-bicyclo[13.3.1]nonadeca-1(19),15,17-trien-13-yl)-butyramide

(2R,4S,5S)-6-(3-Allyloxy-phenyl)-5-[(S)-3-(but-3-ene-1-sulfonyl)-2-methyl-propionylamino]-4-hydroxy-2-methyl-hexanoic acid butylamide (0.220 g, 0.41 mmol) is dissolved in dry dichloromethane (430 ml) under argon and benzylidene(1,3-dimesitylimidazolidin-2-ylidene)(tricyclohexylphosphine)ruthenium dichloride (CAS 246047-72-3) (17 mg, 5 mol %) is added. The reaction is stirred at reflux temperature (bath temperature 60° C.) for 2.5 h. The solvent is evaporated and the residue is purified by chromatography on silica (flashmaster, EtOAc/hexanes 1/1 to EtOAc) followed by a second chromatography on silica (flashmaster, dichloromethane to dichloromethane/MeOH 90/10). The resulting solid is recrystallized from dichloromethane/ether/hexane and then hydrogenated with Pd/C (10% Engelhard 4505, 0.020 g) in methanol (5 ml) at rt at 1 atm hydrogen during 4 h. After filtering through glass wool the solvent is evaporated and the product is purified by preparative TLC (dichloromethane/MeOH 95/5) followed by recrystallization from dichloromethane/ether/hexane to give the product; MS (LC/MS): 533 (M+Na).

1H-NMR (400 MHz, C₂D₂C₁₄): 7.29 (t, 1H), 6.37 (d, 1H), 6.3-6.25 (m, 2H), 5.84 (d, 1H), 5.66 (s, 1H), 4.25-4.15 (m, 3H), 3.90 (d, 1H), 3.80 (s, 1H), 3.35-3.25 (m, 3H), 2.98 (dd, 1H), 2.95-2.75 (m, 4H), 2.70 (dd, 1H), 2.65-2.55 (m, 1H), 2.0-1.6 (m, 8H), 1.6-1.5 (m, 2H), 1.5-1.35 (m, 2), 1.38 (d, 3H), 1.30 (d, 3H), 1.00 (t, 3H).

### EXAMPLE 7

### (2R*,4S*)-N-Butyl-4-hydroxy-2-methyl-4-((S*)-7,7,10-trioxo-2-oxa-7lambda*6*-thia-11-aza-bicyclo[12.3.1]octadeca-1(18),14,16-trien-12-yl)-butyramide

### a) (2R*,4S*,5S*)-6-(3-Allyloxy-phenyl)-4-hydroxy-2-methyl-5-[3-(prop-2-ene-1-sulfonyl)-propionylamino]-hexanoic acid butylamide

[(1S*,2S*,4R*)-1-(3-Allyloxy-benzyl)-4-butylcarbamoyl-2-hydroxy-pentyl]-carbamic acid tert-butyl ester (see example 4b, 0.700 g, 1.56 mmol) is dissolved in 4N hydrochloric acid in dioxane and stirred at rt for 2.5 h. The solvent is evaporated and the residue is dried at low pressure. The residue is redissolved in dry dichloromethane (20 ml). 3-(Prop-2-ene-1-sulfonyl)-propionic acid (see intermediate 4, 0.333 g, 1.87 mmol, 1.2 eq), EDCI (0.448 g, 2.33 mmol, 1.5 eq), HOBt (0.286 g, 1.87 mmol, 1.2 eq) and triethylamine (0.65 ml, 4.7 mmol, 3 eq) are added. The reaction mixture is stirred under argon at rt for 18 h. The mixture is diluted with EtOAc, washed with 0.5N aqueous HCl, sodium bicarbonate solution and brine. Drying over magnesium sulfate, evaporation of the solvents and purification by flash-chromatography on silica (dichloromethane to dichloromethane/MeOH 93/7) gives the product as white solid; MS (LC/MS): 531 (M+Na).

1H-NMR (400 MHz, CDCl3): 7.18 (t, 1H), 6.82-6.72 (m, 3H), 6.16 (d, 1H), 6.10-5.82 (m, 3H), 5.53-5.37 (m, 3H), 5.27 (d, 1H), 4.53 (d, 2H), 4.44 (d, 1H), 4.06 (q, 1H), 3.78-3.63 (m, 3H), 3.38-3.15 (m, 4H), 2.94-2.83 (m, 2H), 2.76-2.47 (m, 3H), 1.53-1.43 (m, 2H), 1.40-1.30 (m, 2H), 1.13 (d, 3H), 0.94 (t, 3H).

### b) (2R*,4S*)-N-Butyl-4-hydroxy-2-methyl-4-((S*)-7,7,10-trioxo-2-oxa-7lambda*6*-thia-11-aza-bicyclo[12.3.1]octadeca-1(18),14,16-trien-12-yl)-butyramide

(2R*,4S*,5S*)-6-(3-Allyloxy-phenyl)-4-hydroxy-2-methyl-5-[3-(prop-2-ene-1-sulfonyl)-propionylamino]-hexanoic acid butylamide (0.370 g, 0.73 mmol) is dissolved in dry dichloromethane (400 ml) under argon and benzylidene(1,3-dimesitylimidazolidin-2-ylidene)(tricyclohexylphosphine)ruthenium dichloride (CAS 246047-72-3) (31 mg, 5 mol %) is added. The reaction is stirred at reflux temperature (bath temperature 60° C.) for 6 h. The solvent is evaporated and the residue is purified by chromatography on silica (dichloromethane to dichloromethane/MeOH 90/10). The resulting solid is hydrogenated with Pd/C (0.050 g, 10% Engelhard 4505) in methanol (30 ml) at rt at 1 atm hydrogen for 19 h. After filtering through glass wool the solvent is evaporated. The residue is dissolved in ethanol and treated with charcoal. Filtration over celite and evaporation gives the product;

MS (LC/MS): 505 (M+Na).

1H-NMR (400 MHz, DMSO): 7.82 (d, 1H), 7.68 (t, 1H), 7.13 (t, 1H), 6.8-6.7 (m, 3H), 4.88 (s, 1H), 4.2-4.05 (m, 2H), 3.85 (t, 1H), 3.42-3.25 (m, 4H), 3.06-2.7 (m, 5), 2.65-2.35 (m, 3H), 1.85-1.6 (m, 6H), 1.4-1.15 (m, 4H), 1.00 (d, 3H), 0.87 (t, 3H).

### EXAMPLE 8

### (2R,4S)—N-Butyl-4-hydroxy-2-methyl-4-((3S,6S)-6-methyl-5,8,8-trioxo-8lambda*6*-thia-4-aza-bicyclo[12.3.1]octadeca-1(17),14(18),15-trien-3-yl)-butyramide

### a) [(S*)-2-(3-Hydroxy-phenyl)-1-((2S*,4R*)-4-methyl-5-oxo-tetrahydro-furan-2-yl)-ethyl]-carbamic acid tert-butyl ester

[(S*)-2-(3-Benzyloxy-phenyl)-1-((2S*,4R*)-4-methyl-5-oxo-tetrahydro-furan-2-yl)-ethyl]-carbamic acid tert-butyl ester (see example 1d, 1.02 g, 2.4 mmol) is hydrogenated (1 atm H2) in ethanol (75 ml) at rt with Pd/C (10% Engelhard 4505, 0.2 g) for 0.5 h. Filtration through a bed of hyflo and evaporation of the solvent gives the product as a white foam.

MS (LC/MS): 358 (M+Na)

### b) Trifluoro-methanesulfonic acid 3-[(S*)-2-tert-butoxycarbonylamino-2-((2S*,4R*)-4-methyl-5-oxo-tetrahydro-furan-2-yl)-ethyl]-phenyl ester

[(S*)-2-(3-Hydroxy-phenyl)-1-((2S*,4R*)-4-methyl-5-oxo-tetrahydro-furan-2-yl)-ethyl]-carbamic acid tert-butyl ester (0.95 g, 2.8 mmol), N-phenyl-bis-(trifluoromethanesulfinimide) (CAS 37595-74-7, 1.01 g, 2.8 mmol, 1.0 eq) and waterfree potassium carbonate (1.17 g, 8.5 mmol, 3 eq) in 10 ml dry THF are heated in a microwave apparatus at 120° C. for 1.5 h. The mixture is diluted with EtOAc and washed with brine. Drying over magnesium sulfate and evaporation of the solvent followed by purification by chromatography on silica (flashmaster, hexane to hexane/EtOAc 60/40) gives the product; MS (LC/MS): 490 (M+Na).

1H-NMR (400 MHz, CDCl3): 7.44 (t, 1H), 7.32 (d, 1H), 7.21-7.19 (m, 2H), 4.57 (d, 1H), 4.52 (t, 1H), 4.06 (q, 1H), 2.99 (d, 2H), 2.81-2.71 (m, 1H), 2.44-2.38 (m, 1H), 1.97-1.90 (m, 1H), 1.40 (s, 9H), 1.31 (d, 3H).

### c) [(S*)-2-(3-Allyl-phenyl)-1-((2S*,4R*)-4-methyl-5-oxo-tetrahydro-furan-2-yl)-ethyl]-carbamic acid tert-butyl ester

Trifluoro-methanesulfonic acid 3-[(S*)-2-tert-butoxycarbonylamino-2-((2S*,4R*)-4-methyl-5-oxo-tetrahydro-furan-2-yl)-ethyl]-phenyl ester (1.01 g, 2.16 mmol) is dissolved in dry DMF. Under argon but-3-enyl-tributyl-stannane (0.75 ml, 2.38 mmol, 1.1 eq), waterfree lithiumchloride (0.23 g, 5.4 mmol, 2.5 eq) and bis-(triphenylphosphin)-palladium(II)-chloride (30 mg, 0.04 mmol, 1.9 mol %) are added and the mixture is stirred at 100° ° C. bath temperature for 45 min. After cooling to rt the mixture is diluted with EtOAc, washed with brine, dried over magnesium sulfate and the solvents are evaporated. The residue is purified by chromatography on silica (flashmaster, hexane to hexane/EtOAc 50/50) followed by crystallization from ether/hexane to give the product as white crystals.

MS (LC/MS): 382 (M+Na)

### d) (S)—N—[(S)-2-(3-Allyl-phenyl)-1-((2S,4R)-4-methyl-5-oxo-tetrahydro-furan-2-yl)-ethyl]-3-(but-3-ene-1-sulfonyl)-2-methyl-propionamide

[(S*)-2-(3-Allyl-phenyl)-1-((2S*,4R*)-4-methyl-5-oxo-tetrahydro-furan-2-yl)-ethyl]-carbamic acid tert-butyl ester (0.32 g, 1.39 mmol) is dissolved in 4N hydrochloride in dioxane (8 ml) and stirred at rt for 1.5 h. The solvent is evaporated and the residue is redissolved in dry dichloromethane (20 ml). (S)-3-(But-3-ene-1-sulfonyl)-2-methyl-propionic acid (see intermediate 1, 0.209 g, 10 mmol, 1.2 eq), EDCI (0.243 g, 1.27 mmol, 1.5 eq), HOBt (0.155 g, 10 mmol, 1.2 eq) and triethylamine (0.35 ml, 2.5 mmol, 3 eq) are added and the reaction mixture is stirred under argon at rt for 18 h. The mixture is diluted with EtOAc, washed with 0.5N aqueous hydrochloric acid, sodium bicarbonate solution and brine, dried over magnesium sulfate and the solvents are evaporated. The two enantiomerically pure diastereomers are separated by chromatography on silica (flashmaster, hexane to hexane/EtOAc 50/50). Mixed fractions are collected and resubmitted to chromatography. The desired compound is the more polar of the two diastereomers (Rf=0.4 in hexane/EtOAc 3/7) and is obtained as yellowish solid; MS (LC/MS): 470 (M+Na);

1H-NMR (400 MHz, CDCl3): 7.25 (t, 1H), 7.13-7.07 (m, 3H), 6.18 (d, 1H), 6.02-5.92 (M, 1H), 5.85-5.75 (m, 1H), 5.20-5.06 (m, 4H), 4.60-4.53 (m, 1H), 4.37 (q, 1H), 3.42-3.35 (m, 3H), 3.03-2.83 (m, 6H), 2.73-2.64 (m, 1H), 2.60-2.53 (m, 2H), 2.32-2.25 (m, 1H), 1.93-1.86 (m, 1H), 1.33 (d, 3H), 1.27 (d, 3H).

### e) (2R,4S)—N-Butyl-4-hydroxy-2-methyl-4-((3S,6S)-6-methyl-5,8,8-trioxo-8lambda*6*-thia-4-aza-bicyclo[12.3.1]octadeca-1(17),14(18),15-trien-3-yl)-butyramide

(S)—N—[(S)-2-(3-Allyl-phenyl)-1-((2S,4R)-4-methyl-5-oxo-tetrahydro-furan-2-yl)-ethyl]-3-(but-3-ene-1-sulfonyl)-2-methyl-propionamide (140 mg, 0.31 mmol) is dissolved in dry dichloromethane under argon and benzylidene(1,3-dimesitylimidazolidin-2-ylidene)(tricyclohexylphosphine)ruthenium dichloride (CAS 246047-72-3) (13 mg, 5 mol %) is added. The reaction is heated at reflux temperature (bath temperature 60° C.) for 2.5 h. The solvent is evaporated and the residue is purified by chromatography on silica (flashmaster, hexane/EtOAc 80/20 to EtOAc) to give the ring-closed intermediate (3S,6S)-6-Methyl-3-((2S,4R)-4-methyl-5-oxo-tetrahydro-furan-2-yl)-8,8-dioxo-8lambda*6*-thia-4-aza-bicyclo[12.3.1]octadeca-1(18),11,14,16-tetraen-5-one as a grayish solid (98 mg, 0.23 mmol). This is dissolved in butylamine (5 ml) and stirred for 18 h at reflux temperature (bath temperature 90° C.). The solvent is evaporated and the residue is purified by chromatography on silica (flashmaster, dichloromethane to dichloromethane/MeOH 92/8) and crystallization from dichloromethane/ether/hexane to give 58 mg white crystals. They are dissolved in methanol (5 ml) and hydrogenated (1 atm H2) at rt with Pd/C (10% Engelhard 4505, 30 mg) for 5 h. Purification by chromatography on silica (flashmaster, dichloromethane to dichloromethane/MeOH 90/10) and crystallization from dichloromethane/ether/hexane gives the product as white crystals; MS (LC/MS): 517 (M+Na);

1H-NMR (400 MHz, CDCl3): 7.26 (t, 1H), 7.10-7.06 (m, 3H), 5.96 (d, 1H), 5.81 (t, 1H), 4.39 (t, 1H), 3.95-3.90 (m, 1H), 3.35-3.25 (m, 3H), 3.03-2.68 (m, 2H), 2.8-2.4 (m, 7H), 1.8-1.45 (m, 9H), 1.45-1.25 (m, 4H), 1.32 (d, 3H), 1.30 (d, 3H), 0.96 (t, 3H).

### EXAMPLE 9

### (2R,4S)—N-Butyl-4-hydroxy-2-methyl-4-((3S,6S)-6-methyl-5,8,8-trioxo-8lambda*6*-thia-4-aza-bicyclo[13.3.1]nonadeca-1(18),15(19),16-trien-3-yl)-butyramide

The title compound is obtained in analogues manner as described for example 8, starting from [(S*)-2-(3-Allyl-phenyl)-1-((2S*,4R*)-4-methyl-5-oxo-tetrahydro-furan-2-yl)-ethyl]-carbamic acid tert-butyl ester (see example 8c) and (S)-2-methyl-3-(pent-4-ene-1-sulfonyl)-propionic acid (see intermediate 3a); MS (LC/MS): 531 (M+Na),

1H-NMR (400 MHz, CDCl3): 7.24 (t, 1H), 7.10-7.04 (m, 3H), 6.00 (d, 1H), 5.84 (t, 1H), 4.37 (t, 1H), 3.95-3.87 (m, 1H), 3.61 (dd, 1H), 3.31-3.26 (m, 2H), 3.02 (d, 1H), 2.92-2.85 (m, 1H), 2.82-2.76 (m, 1H), 2.68-2.55 (m, 4H), 2.34-2.22 (m, 2H), 1.78-1.58 (m, 6H), 1.56-1.48 (m, 2H), 1.43-1.16 (m, 7H), 1.34 (d, 3H), 1.29 (d, 3H), 0.96 (t, 3H).

### Intermediate 1: (S)-3-(But-3-ene-1-sulfonyl)-2-methyl-propionic acid

### a) (S)-3-But-3-enylsulfanyl-2-methyl-propionic acid

(S)-3-Acetylsulfanyl-2-methyl-propionic acid (5.0 ml, 36 mmol) and 4-bromo-but-1-ene (3.75 ml, 36 mmol, 1.0 eq) are dissolved in MeOH (55 ml) and 4 N sodiumhydroxide in water (27.2 ml, 108 mmol, 3 eq) are added under initial cooling. The mixture is stirred at rt for 2 h. Aqueous 1N hydrochloric acid (90 ml) is added and the mixture is extracted with EtOAc. Washing with water and brine, drying over magnesium sulfate and evaporation of the solvent gives the crude product that is purified by chromatography on silica (flashmaster, hexane to hexane/EtOAc 40/60). The product is obtained as yellowish oil.

### b(S)-3-(But-3-ene-1-sulfonyl)-2-methyl-propionic acid

(S)-3-But-3-enylsulfanyl-2-methyl-propionic acid (1.0 g, 5.74 mmol) is dissolved in water (5 ml) and acetonitrile (10 ml) and cooled to −10° C. Oxone (3.49 g 47%, 23 mmol, 4 eq) is added in portions, keeping the temperature below 0° C. The reaction is then stirred without cooling for 36 h. The mixture is diluted with water and extracted with EtOAc. The organic layer is washed with water and brine, dried over magnesium sulfate and the solvent is evaporated. The crude product is purified by chromatography on silica (flashmaster, dichloromethane to dichloromethane/MeOH 90/10). Tituration with cold hexane gives the product as white crystals; MS (LC/MS): 229 (M+Na).

### Intermediate 2: (S)-2-Methyl-3-(prop-2-ene-1-sulfonyl)-propionic acid

(S)-2-Methyl-3-(prop-2-ene-1-sulfonyl)-propionic acid is obtained in analogous manner as described for intermediate 1, starting from (S)-3-acetylsulfanyl-2-methyl-propionic acid and 3-bromo-prop-1-ene.

### Intermediate 3: 2-Methyl-3-(pent-4-ene-1-sulfonyl)-propionic acid

2-Methyl-3-(pent-4-ene-1-sulfonyl)-propionic acid is obtained in analogous manner as described for intermediate 1, starting from 3-acetylsulfanyl-2-methyl-propionic acid and 5-bromo-pent-1-ene.

### Intermediate 3a: (S)-2-Methyl-3-(pent-4-ene-1-sulfonyl)-propionic acid

(S)-2-Methyl-3-(pent-4-ene-1-sulfonyl)-propionic acid is obtained in analogous manner as described for intermediate 1, starting from (S)-3-acetylsulfanyl-2-methyl-propionic acid and 5-bromo-pent-1-ene.

### Intermediate 4: 3-(Prop-2-ene-1-sulfonyl)-propionic acid

### a) 3-Allylsulfanyl-propionic acid

3-Mercapto-propionic acid (10.0 ml, 115 mmol) and 3-bromo-propene (9.72 ml, 115 mmol, 1.0 eq) are dissolved in methanol (28 ml) and cooled in an icebath. 4N aqueous sodium hydroxid solution (86.2 ml, 0.34 mmol, 3 eq) is added and the mixture is stirred at rt for 6 h. 1N aqueous hydrochloric acid is added until acidic pH and the mixture is extracted with EtOAc. Washing with water and brine, drying over magnesium sulfate and evaporation of the solvent gives the product as a yellow oil.

### b) 3-(Prop-2-ene-1-sulfonyl)-propionic acid

3-Allylsulfanyl-propionic acid (0.94 g, 6.4 mmol) is dissolved in water (10 ml) and acetonitrile (20 ml) and cooled to −10° C. Oxone (19.8 g 47%, 32 mmol, 5 eq) is added in portions, keeping the temperature below 0° C. The reaction is then stirred without cooling for 8 h. The precipitate is filtered off and washed with acetonitrile. The filtrate is evaporated at reduced pressure and then distributed between water and EtOAc. The organic layer is washed with water and brine and dried over magnesium sulfate. Evaporation of the solvent gives the product as a white solid.

1. A compound of formula I 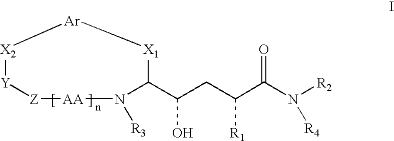
wherein
R₁ is (C₁₋₈)alkyl, (C₁₋₄)alkoxy(C₁₋₄)alkyl, hydroxy(C₁₋₆)alkyl, (C₁₋₄)alkylthio(C₁₋₄)alkyl, (C₁₋₆)alkenyl, (C₃₋₇)cycloalkyl, (C₃₋₇)cycloalkyl(C₁₋₄)alkyl, piperidinyl or pyrrolidinyl,
R₂ and R₄, independently, are hydrogen or optionally substituted (C₁₋₈)alkyl, (C₃₋₇)cycloalkyl, (C₃₋₇)cycloalkyl(C₁₋₄)alkyl, aryl, aryl(C₁₋₄)alkyl, heteroaryl or heteroaryl(C₁₋₄) alkyl, or
R₂ and R₄, together with the nitrogen to which they are attached, form an optionally substituted piperidino, pyrrolidinyl, morpholino or piperazinyl group,
R₃ is hydrogen or (C₁₋₄)alkyl,
X₁ is CH₂,
X₂ is CH₂, O, S, CO, COO, OCO, NHCO, CONH, or NR, R being hydrogen or (C₁₋₄)alkyl,
Y is (C₁₋₈)alkylen or (C₁₋₈)alkylenoxy(C₁₋₆)alkylen, (C₁₋₈)alkenylen or (C₁₋₈)alkenylenoxy(C₁₋₆)alkylen,
Ar is a phenyl ring optionally mono- di- or trisubstituted by, independently, hydroxy or halogen, whereby X₁ and X₂ are in meta or para position to each other,
and either
Z is CO,
AA is a natural or unnatural alpha-amino-acid, and
n is 0 or 1,
or
Z is SO₂,
AA is an optionally substituted ethylencarbonyl group (derived from a natural or unnatural alpha-amino acid by replacement of the nitrogen by a methylen group), and
n is 1
in free base or acid addition salt form.

2. A compound of formula I according to claim 1, wherein
R₁ is (C₁₋₈)alkyl, (C₁₋₄)alkoxy(C₁₋₄)alkyl, hydroxy(C₁₋₆)alkyl, (C₁₋₄alkylthio(C₁₋₄)alkyl, (C₁₋₆)alkenyl, (C₃₋₇)cycloalkyl, (C₃₋₇)cycloalkyl(C₁₋₄)alkyl, piperidinyl or pyrrolidinyl,
R₂ and R₄, independently, are
(a) hydrogen
(b) (C₁₋₈)alkyl, (C₃₋₇) cycloalkyl or (C₃₋₇)cycloalkyl(C₁₋₄)alkyl, in each case optionally substituted by one to three groups selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonylamino, (C₁₋₄)alkylcarbonyl, (C₁₋₄)sulfonyl, cyano, oxo, hetero (C₃₋₇)cycloalkyl or heteroaryl, or
(c) aryl, aryl(C₁₋₄)alkyl, heteroaryl or heteroaryl(C₁₋₄)alkyl, wherein in the latter two radicals heteroaryl denotes an aromatic 5- or 6-membered ring in which 1, 2 or 3 atoms are heteroatoms independently selected from O, N and S, wherein all radicals are optionally substituted by one to three groups selected from halogen, hydroxy, cyano, trifluoromethyl, carboxy, (C₁₋₄)alkyloxycarbonyl, (C₁₋₄)alkylcarbamoyl, (C₁₋₄)alkylsulfonyl, (C₁₋₄)alkylcarbonyloxy, (C₁₋₄)alkylcarbonyl, (C₁₋₄)alkyl, (C₁₋₄)alkoxy or hydroxy(C₁₋₄)alkyl, or
R₂ and R₄, together with the nitrogen to which they are attached, form an piperidino, pyrrolidinyl, morpholino or piperazinyl group, each of which is optionally substituted by one to three groups selected from hydroxy, hydroxy(C₁₋₄)alkyl, (C₁₋₄)alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonylamino, (C₁₋₄)alkylcarbonyl, (C₁₋₄)sulfonyl, cyano, oxo, hetero (C₃₋₇)cycloalkyl or heteroaryl,
R₃ is hydrogen or (C₁₋₄)alkyl,
X₁ is CH₂,
X₂ is CH₂, O, S, CO, COO, OCO, NHCO, CONH, or NR, R being hydrogen or (C₁₋₄)alkyl,
Y is (C₁₋₈)alkylen or (C₁₋₈)alkylenoxy(C₁₋₆)alkylen, (C₁₋₈)alkenylen or (C₁₋₈)alkenylenoxy(C₁₋₆)alkylen,
Ar is a phenyl ring optionally mono- di- or trisubstituted by, independently, hydroxy or halogen, whereby X₁ and X₂ are in meta or para position to each other,
and either
Z is CO,
AA is a natural or unnatural alpha-amino-acid, and
n is 0 or 1,
or
z is SO₂,
AA is an optionally substituted ethylencarbonyl group (derived from a natural or unnatural alpha-amino acid by replacement of the nitrogen by a methylen group), and
n is 1
in free base or acid addition salt form.

3. A compound of formula I according to claim 1, wherein
R₁ is (C₁₋₄)alkyl,
R₂ is (C₁₋₆)alkyl,
R₃ is hydrogen or (C₁₋₄)alkyl,
R₄ is hydrogen,
X₁ is CH₂,
X₂ is CH₂ or O,
Y is (C₁₋₈)alkylen,
Ar is unsubstituted phenylen, whereby X₁ and X₂ are in meta position to each other,
and either
Z is CO,
AA is a natural or unnatural alpha-amino-acid, and
n is 0 or 1,
or
Z is SO₂,
AA is an optionally substituted ethylencarbonyl group (derived from a natural or unnatural alpha-amino acid by replacement of the nitrogen by a methylen group), and
n is 1
in free base or acid addition salt form.

4. A compound of formula I according to claim 1, wherein
R₁ is (C₁₋₄)alkyl,
R₂ is (C₁₋₆)alkyl,
R₃ is hydrogen or (C₁₋₄)alkyl,
R₄ is hydrogen,
X₁ is CH₂,
X₂ is CH₂ or O,
Y is (C₃₋₆)alkylen,
Ar is unsubstituted phenylen whereby X₁ and X₂ are in meta position to each other,
and either
Z is CO,
AA is —N(H)—CH(CH₃)ₘ—C(O)—, wherein m is 0 or 1 and
n is 0 or 1,
or
Z is SO₂,
AA is —CH₂—CH(CH₃)—C(O)— or —CH₂—CH₂—C(O)— and
n is 1
in free base or acid addition salt form.

5. A process for the preparation of a compound of formula I as defined in claim 1, or a salt thereof, which includes the steps of
a) for the production of a compound of formula I wherein Z is CO, cyclisation by amide formation of a compound of formula II 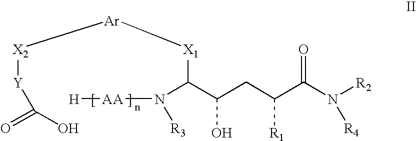
wherein R₁, R₂, R₃, R₄, X₁, X₂, Y, Ar, AA and n are as defined in claim 1,
b) for the production of a compound of formula I wherein Z is SO₂ and Y is (C₁₋₈)alkenylen or (C₁₋₈)alkenylenoxy(C₁₋₆)alkylen, cyclisation by metathesis of a compound of formula III 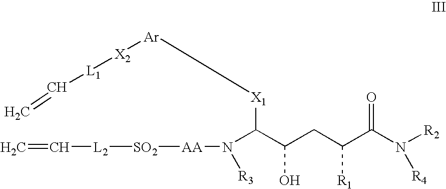
wherein R₁, R₂, R₃, R₄, X₁, X₂, Ar and AA are as defined in claim 1 and L₁ and L₂, independently are alkylen or alkylenoxyalkylen groups, or
c) for the production of a compound of formula I wherein Z is SO₂ and Y is (C₁₋₈)alkylen or (C₁₋₈)alkylenoxy(C₁₋₆)alkylen, hydrogenation of a compound of formula I wherein Z is SO₂ and Y is (C₁₋₈)alkenylen or (C₁₋₈)alkenylenoxy(C₁₋₆)alkylen,
and recovering the so obtained compound of formula I in free base or acid addition salt form.

6. A compound according to claim 1 in free base or pharmaceutically acceptable acid addition salt form, for use as a pharmaceutical.

7. A compound according to claim 1 in free base or pharmaceutically acceptable acid addition salt form, for use in the treatment of neurological and vascular disorders related to beta-amyloid generation and/or aggregation.

8. A pharmaceutical composition comprising a compound according to claim 1 in free base of pharmaceutically acceptable acid addition salt form, in association with a pharmaceutical carrier or diluent.

9. (canceled)

10. (canceled)

11. A method for the treatment of neurological and vascular disorders related to beta-amyloid generation and/or aggregation in a subject in need of such treatment, comprising:
administering to such subject a therapeutically effective amount of a compound according to claim 1 in free base or pharmaceutically acceptable acid addition salt form.

12. A pharmaceutical combinations comprising
a therapeutically effective amount of a compound according to claim 1 in free base of pharmaceutically acceptable acid addition salt form and a second drug substance, for simultaneous or sequential administration.
